(19) United States
(12) Patent Application Publication
(10) Pub. No.: US 2024/0417673 A1
(43) Pub. Date: Dec. 19, 2024

(54) GIANT ORGANELLES RECOVERY AND USE THEREOF

(71) Applicants: PARIS SCIENCES ET LETTRES, Paris (FR); ÉCOLE NORMALE SUPÉRIEURE, Paris (FR); CENTRE NATIONAL DE LA RECHERCHE SCIENTIFIQUE, Paris (FR); SORBONNE UNIVERSITÉ, Paris (FR); UNIVERSITÉ PARIS CITÉ, Paris (FR)

(72) Inventors: Abdou Rachid THIAM, Paris (FR); Alexandre SANTINHO, Paris (FR); Vincent FAUGERAS, Paris (FR)

(21) Appl. No.: 18/700,200

(22) PCT Filed: Oct. 13, 2022
(86) PCT No.: PCT/EP2022/078560

§ 371 Date: Apr. 10, 2024

(30) Foreign Application Priority Data
Oct. 13, 2021 (EP) 21202428.5

(51) Int. Cl. C12N 1/06; G01N 33/50

(52) U.S. Cl. CPC C12N 1/066; G01N 33/5076; C12N 2509/10

(57) ABSTRACT

A method based on the combination of a particular osmotic swelling, an adapted and controlled plasma membrane lysis and removal, to produce the giant extracellular organelles vesicles, and use of the giant extracellular organelles vesicles to screen the activity of proteins or exogenous molecules, the method includes: contacting the cells during 0.5 to 30 minutes with an hypotonic aqueous medium with an osmolarity ranging from 0.1 to 100 mOsm/L; applying a membrane tension on cells ranging from 10⁻³ to 5 mN/m during 10⁻⁴ to 100 seconds; and collecting the giant extracellular organelle vesicles into the hypotonic aqueous medium.

### TECHNICAL FIELD OF THE INVENTION

The present invention concerns a new method based on the combination of an osmotic swelling by immersion of cells in specific hypotonic aqueous media to generate over-micrometric swollen intracellular organelles having an increased surface-to-volume ratio, followed by an adapted cell membrane lysis to isolate and recover stable and functional giant extracellular organelle vesicles (GEOVs) from cells, and use of such giant extracellular organelle vesicles for several applications including the screening of the activity of proteins or exogenous molecules or drugs.

### STATE OF THE ART

A major goal and limitation in cell biology, biochemistry, drug discovery, pathogenesis, is to know what happens to each organelle upon a given environmental change. It is important to know where proteins are exactly localized, how they get recruited and impact organelle properties, what are their functions. Intracellular proteins such as ion channels or enzymes constitute for instance essential pharmacological drug targets. These limitations could be circumvented if organelles (mostly bilayer-bounded compartment) were over few micrometers in size and modulable outside their hosting cells.

Current methods to extract intracellular fragments are often based on the recovery of cell membrane fragments through the combination of lysis buffers (hypotonic buffers, buffers+detergent molecules) followed by a homogenization step resulting in the obtention of nanofragments (hundreds of nanometers sized). For this method, no optical visualization and manipulation is possible so long purification steps are needed to identify the presence of the desired nanofragments of organelles, which makes industrial applications difficult. Cell surgery has also been practiced a lot with optical tweezers, lasers, AFM, pipette suction, in order to extract one micro-fragment of an organelle (mitochondria, lysosome, nucleus). Such techniques do not allow the collection of micrometric organelles for each type of organelles, and it allows the collection of only one organelle per cell at a time, which makes industrial applications difficult.

Recent studies have shown that submitting 2D cultured-cells to a hypotonic buffer during few minutes leads to the swelling of organelles, without detaching cells from their support. In these experiments, the cells remained intact and are still attached to their support and, also among them, thanks to their extracellular matrix. Thus, the organelles inside are confined and do not exhibit their whole potential in terms of size and accessibility, lacking space in 2D attached cells (support-attached swell less efficiently than the ones that can detach). The organelles were also trapped because of the high difficulty to break the cell plasma membrane and have it fully removed without breaking the organelles into nanofragments.

Some companies selling cell lysis kit, developed a protocol by which a slight hypotonic medium used with detergents followed by a shear induced on cells at high pressure (>1 bar) with macroscopic syringes (>0.5 mm in radius) lead to cell lysis (https://www.thermofisher.com/sn/en/home/life-science/protein-biology/protein-biology-learning-center/protein-biology-resource-library/pierce-protein-methods/traditional-methods-cell-lysis.html; https://www.sigmaaldrich.com/FR/fr/product/sigma/mcl1?gclid=Cj0KCQjw0K-HBhDDARIsAFJ6UGgiWEUGiKbual-z_q3z21w7hBtnBD_z3iEBRTtuqNDZ4NRz_rFxdXwaAgM5EALw_wcB). These kits and classic protocols do not form giant intracellular organelle vesicles before extraction: Their swelling protocol is not sufficient to generate stable and functional giant organelle vesicles and their shearing process, to break the plasma membrane, is so strong that the organelles also break apart into extracellular nanofragments of organelles with potential loss of biochemical composition (luminal and membrane proteins can be loosed during the process), activity, functionality and/or membrane biophysical properties.

Thus, there is still a need to generate, extract and recover stable and functional giant organelles from cells, free from their source cytosol, not surrounded by plasma membrane, and sufficiently large (e.g. 5-20 μm) for both industrial and academic manipulation purpose, otherwise called giant extracellular organelle vesicles, from any cells.

### DESCRIPTION OF THE INVENTION

Therefore, the Inventors established a method based on a particular combination of osmotic, biological and physico-chemical perturbations and plasma membrane-targeted breaking approaches to generate, extract and recover stable and functional giant organelles from cells, i.e. to produce giant extracellular organelle vesicles (GEOVs). To this aim the Inventors have developed a method based on both (i) a step of osmotic swelling to generate intracellular organelles having an increased surface-to-volume ratio compared to their original form, where cells are put in contact with a hypotonic aqueous solution with an osmolarity ranging from 0.1 to 100 mOsm/L, preferably from 1 to 50 mOsm/L, more preferably from 5 to 50 mOsm/L, the most preferably between 10 mOsm/L to 40 mOsm/L, during 0.5, 3, 5, 7, 10, 15, 20 to 30 minutes; the aqueous media is eventually combined with chemicals, to increase the size of intracellular organelle vesicles (bilayer-bounded compartments) leading to the formation of enlarged vesicle of a size distribution never reported in the prior literature, called giant (intracellular) organelle vesicles (GIOVs) and, to reduce the plasma membrane lysis tension value; this step is eventually followed by (ii) a step where a back-and-forth motion of the hypotonic aqueous medium is generated to displace the cells at a speed ranging from 0.01 m/s to 10 m/s during 0.01 seconds to 10 minutes; this step promotes the disruption of both the cytoskeleton and the extracellular matrix of the cells, in order to reduce the plasma membrane lysis tension and provoke plasma membrane full opening following lysis; then (iii) a step where cells' plasma membrane tension is increased (from 10⁻³ to 5 mN/m, preferably from 5·10⁻³ to 4 mN/m, more preferably from 10⁻² to 2 mN/m, even more preferably from 10⁻² to 1 mN/m most preferably between 5·10⁻² to 0.75 mN/m) mechanically from cells of step (i) or steps (i)+(ii) during 10⁻⁴ to 100 seconds leading to the lysis and full removal of their plasma membrane and the release in the hypotonic medium of stable and functional giant extracellular organelle vesicles (GEOVs), without lysing them, with a mean size generally ranging from about 5 to 20 μm, with a surface-to-volume ratio (surface divided by the volume of a geometric shape, spherical in most of the case) ranging from 3 μm⁻¹ to 0.15 μm⁻¹, preferably from 2 μm⁻¹ to 0.15 μm⁻¹, more preferably from 1.5 μm⁻¹ to 0.15 μm⁻¹, most preferably between 1.2 μm⁻¹ to 0.15 μm⁻¹. For example, GEOVs have an increased surface-to-volume ratio which generally ranges from 1.2 μm⁻¹ (for a size >5 μm) to 0.3 μm⁻¹ (for a size <20 μm). To sum up, in the whole protocol, the strong osmotic shock allows generate giant intracellular vesicles and to specifically destabilize the plasma membrane of cells, which then only needs a small mechanical perturbation to fully break and release the giant extracellular organelle vesicles, which are not broken thanks to the protocol we developed for the plasma membrane lysis and removal.

According to the present invention, the term “giant extracellular organelle vesicles” (GEOVs) means swollen bilayer-bounded organelles generated, extracted and collected from its hosting cell, as vesicles (with a lumen being bilayer-bounded), freed from the plasma membrane which surrounded it; having a mean surface-to-volume ratio S/V ranging from 3 μm⁻¹ to 0.15 μm⁻¹, preferably from 2 μm⁻¹ to 0.15 μm⁻¹, more preferably from 1.5 μm⁻¹ to 0.15 μm⁻¹, the most preferably between 1.2 μm⁻¹ to 0.15 μm⁻¹. This corresponds to a mean size ranging from about 2 to 40 μm, preferably from 3 to 40 μm, more preferably from 4 to 40 μm, the most preferably from 5 to 40 μm. Such giant extracellular organelle vesicles derive from organelles from the group consisting of endoplasmic reticulum, mitochondria, lysosome, Golgi apparatus, vacuole, chloroplast, autophagosome, autolysosomes, endosomes, peroxisome, multivesicular bodies. Both membrane and lumen of such giant extracellular organelle vesicles are composed of proteins, lipids and metabolites. The total amount of biochemical material of such giant extracellular organelle vesicles is a fraction of the total composition of the organelle from which they are produced. This fraction is, at least, larger than 0.01%, preferably larger than 0.1%, more preferably larger than 1%, the most preferably larger than 10%. Such giant extracellular organelle vesicles are produced having none, one, or several contacts with other GEOVs. Such Giant Organelles vesicles are produced having none, one, or several contacts with cell components such as cytosol biomolecules, microtubules, actin, filaments, cell nucleus, lipid droplets, ribosomes, centrioles, plasma membrane.

The giant extracellular organelle vesicles of the present invention make it now possible to turn toward applications previously unthinkable for industrial players. In addition, contact between such giant organelle vesicles can be conserved. Another advantage is the rapid isolation of giant organelle vesicles easily imaged with a microscope (e.g. about 5 to 20 μm sized), which, for example, allows a fast quantification of drug interaction and/or effect on protein activity. This facilitates the process compared to other techniques from the art which have to concentrate the fragments of organelles and then do some biochemistry quantification.

Such giant extracellular organelle vesicles are functional, can be labelled, have any protein wanted on them, and can be easily picked and manipulated. This technology thus enables production of giant extracellular organelle vesicles that could be used for several applications including the screening of membrane/luminal proteins activity and/or the effect of exogenous molecules or drugs. In the field of pharmaceutical R&D, this activity can be measured with or without the presence of a drug. It concerns membrane and soluble proteins or enzymes, e.g. ions channels, ribosomes, etc. . . . More globally, this technology offers for the first time the possibility to map drug-organelle interactions to establish the selectivity/specificity and/or toxicity of drug candidate interacting with proteins localized to a given organelle.

The present invention thus relates to a method for production of giant extracellular organelle vesicles from any cells, said method comprising:
a) Contacting the cells during 0.5 to 30 minutes with an hypotonic aqueous medium with an osmolarity ranging from 0.1 to 100 mOsm/L, for swelling both cells and its intracellular bilayer-bounded organelles, disrupting cell both cytoskeleton and extracellular matrix, spherizing the cell;
b) Applying a membrane tension on cells ranging from 10⁻³ to 5 mN/m, during 10⁻⁴ to 100 seconds, for lysis and the removal of cell plasma membranes;
c) Recovery of giant extracellular organelle vesicles in the hypotonic aqueous medium.

According to a particular embodiment of the method of the present invention, the cells from which the giant extracellular organelle vesicles are obtained can be cultured on a support or in suspension in bulk in any appropriate medium well known from the art, or can be recovered from organoids, tissues, organs or organisms. Any type of cells (mammalian, plant, or bacteria cells) can be used to generate giant organelles according to the method of the present invention. For example, cells derived from Cos 7, Huh, mammalian cells, human cells, tumor cells, obtained from the market, laboratories and hospital patients.

According to a particular embodiment of the method of the present invention, step a) is performed using a hypotonic aqueous medium which is any aqueous solution with an osmolarity ranging from 0.1 to 100 mOsm/L, preferably from 1 to 50 mOsm/L, more preferably from 5 to 50 mOsm/L, the most preferably from 10 to 40 mOsm, during 0.5, 3, 5, 7, 10, 15, 20 to 30 minutes, derived from, for example, buffer solutions (Diluted DPBS), diluted cell culture medium (DMEM), ionic solutions, salt solutions (e.g. CaCl₂) or KCl solutions), diluted buffer, water, etc. . . . , for the generation of stable and functional giant organelle vesicles. Thus for example, for COS-7 cells, the cytoplasm osmolarity is around 300 mOsm/L, meaning that all kind of aqueous solutions with an osmolarity below about 100 mOsm/L and higher than 0.1 mOsm/L, can be used to generate giant organelle vesicles. Such hypotonic aqueous medium enables to instantly apply to cells and its intracellular organelles an adequate non-destructive, fast and effective osmotic shock to generate spherical swollen cells and enlarged organelle vesicles. Otherwise, the swelling protocols are too long and not effective, leading to non-spherical compartments and protein degradation, which makes the production of GEOVs impossible. The control of the swelling kinetics parameters is essential for the production of giant extracellular organelle vesicles.

Some added molecules (cytoskeleton disruptor such as nocodazole, navelbine, latrunculin A, latrunculin B, cytochalasin; kinesin, myosin and dinein motor inhibitors such as blebistatin, benzytoluen sulphonamide, butanediome monoxime) also allow to disassemble the cytoskeleton and reduce the lysis tension of the cells. Such hypotonic aqueous medium ensures that in the majority of cases, the intracellular compartments will form, after cell lysis, giant extracellular spherical vesicles from the organelles with a size and surface-to-volume ratio never reached before by the processes of the prior art. Thus, the hypotonic medium of the method of the present invention can also contain one or more molecules to modulate the surface-to-volume ratio distribution of giant extracellular organelle vesicles while preventing their degradation and the value of surface tensions to lyse the plasma membrane (e.g. protease inhibitors, molecular motors inhibitors, organelle-cytoskeleton contact inhibitors, cytoskeleton disruptors, detergents). Some added molecules (e.g. ion channels modulator/blocker: thaspsigargin, caffein, benzothiasepin; Extra-cellular matrix distruptor such as trypsin; Protein transport inhibitors; Protein signaling inhibitors such as xelospongin; Chemical detergents such as Triton-X-100, octyl-glucoside, DDM, carboxylic acids) allow the cells to swell faster, and therefore to decrease the surface-to-volume ratio of produced giant extracellular organelle vesicles more quickly. Finally, the cell swelling kinetics is crucial not only to decrease the input energy to lyse the cell plasma membrane of step b) but also to control the size of the GEOVs produced at the end of step c).

According to a particular embodiment of the method of the present invention, the hypotonic aqueous medium comprises one or more molecules chosen from the group consisting of: nocodazole, latrunculins, trypsin, misakinolides, mycalolides, aplyronides, vinblastine, rotenone, swinholides, jasplakinolides, vincristine, demecolcine, cytochalasins, colchicine, vinca-alcaloids, dihydropyridine, phenylalkylamine, benzothiazepine, gabapentinoids, blebistatin, benzytoluen sulphonamide, butanediome monoxime, thaspsigargin, xelospongin, Triton X-100, Tween, SDS, Brij, Octyl Glucoside, octyl thioglucoside, CHAPS, CHAPSO, magnesium, said molecules being added before step a), at step a), a′), b) and/or c).

According to a particular embodiment of the method of the present invention, prior to the generation of giant extracellular organelle vesicles, cells may be treated with molecules to prevent protein degradation, modulate biochemical reactions on organelles, etc. . . . so that giant extracellular organelle vesicles can bear specific biochemical properties. The metabolic conditions and the architecture of cells and its organelles can thus be further modified before step b), by chemicals and/or by modifying the expression level of proteins impacting the architecture of cells and/or their organelles and/or the metabolic conditions. Indeed prior to step a), cells can be treated with chemicals or can be modified to overexpress or repress the level of some proteins that impact the architecture of the cell and its organelles-notably the surface-to-volume ratio and the relative positioning of the organelles with each other—and the metabolic conditions. These manipulations enable controlling the size of the recovered giant extracellular organelle vesicles and their contact: tuning the surface-to-volume ratio, prior to the osmotic swelling, impacting the future recovered giant organelle size, with adapted osmotic swelling. For example, prior to inducing the osmotic swelling, the surface-to-volume ratio of the future produced giant extracellular organelles vesicles can be modulated (1) by altering the expression levels of proteins impacting organelle shape and contact sites with other organelles, plasma membrane and cytoskeleton, (2) by treating cells with molecules that alter: both the cytoskeleton and molecular motors, organelle contacts sites (with other organelles, plasma membrane and cytoskeleton), molecules-transporting-proteins activity localized on plasma membranes and organelles, signaling protein activity (3) by altering cellular metabolic pathways impacting organelles number and shape, surface, (4) any treatment mediate change in organelle surface-to-volume ratio and inter-organelle contact. For example, overexpressing climp63 prior to the generation and recovery of giant extracellular organelles vesicles leads to larger giant extracellular organelles vesicles emanating from the endoplasmic reticulum with sizes larger than 30 μm. In the same way, overexpressing Mfn2 prior to the generation and recovery of giant extracellular organelles vesicles leads to larger giant extracellular organelles vesicles emanating from the mitochondria with surface-to-volume ratio smaller than 0.75 μm⁻¹. Pre-treating cells with nocodazole (or Latrunculin A) between 1 and 90 min before swelling allows the formation of bigger giant organelles from the ER. Adding rapamycin between 12 to 24 hours before swelling formation allows to form bigger giant extracellular organelles vesicles from endosome, lysosome, autolysosome and multivesicular body. Adding bafylomicin before swelling and extraction also allows to obtain more giant extracellular organelles vesicles coming from autophagosomes.

According to a particular embodiment of the method of the present invention, said method further comprises, after step a) and before step b), a step a′) comprising generating a back-and-forth motion of the hypotonic aqueous medium to displace cells at a speed ranging from about 0.01 m/s to 10 m/s during about 0.01 seconds to 10 minutes, to disrupt both cytoskeleton and extracellular matrix of cells. This optional step a′) applied to swollen cells of step a) thus allows to further apply a less important stretching membrane tension in step b) to lyse cells, open them completely and release GEOVs without lysing them. This is because the cytoskeleton gives the plasma membrane of the cell an additional resistance. Thus, when swollen cells and organelles from step a) are not subjected to such a motion, the average lysis tension needed in step b) is about ˜7 mN/m (some cells lyse at under ˜1 mN/m and other at ˜10 mN/m, as shown in example), whereas for swollen cells and organelles from step a) subjected to such motion in step a′), the average lysis tension needed in step b) is about ˜2 mN/m (A large majority of cells lyses at tension under 1 mN/m, as shown in example). Therefore, subjecting the cells to motion, without lysing them, allows to lower the further lysis tension without damaging the giant (intracellular) organelle vesicles still inside. In the literature, membrane rupture is almost always associated with cell lysis. In the method of the present invention, cell lysis leads to the release of all or almost all of the cell's contents (including organelles) into the extracellular medium. The state of the art does not show what happens after membrane rupture and misuse the term cell lysis because there is no release of intracellular compounds. Indeed, it is extremely common that plasma membrane rupture leads to vesiculation or to the opening of a bilayer pore which closes. In these cases, the organelles are never released although the state of the art is mentioning the term of plasma membrane rupture and/or cell lysis measurement. This is not because there is membrane rupture and/or cell lysis, that the intracellular content is released. The method of the present invention allows to drastically decrease (≤2 mN/m) the membrane tension to reached cell lysis, the removal the whole plasma membrane and the release of all or almost all GEOVs from a cell (as shown in example 1), without lysing them thanks to gentle lysis.

According to a particular embodiment of the method of the present invention, step b) is performed to apply a stretching membrane tension (bilayer tension) on cells ranging from 10⁻³ to 5 mN/m, preferably from 5·10⁻³ to 4 mN/m, more preferably from 10⁻² to 2 mN/m, even more preferably from 10⁻² to 1 mN/m most preferably between 5·10⁻² mN/m to 0.75 mN/m, during 10⁻⁴ to 100 seconds, so as to break cell plasma membranes rapidly while preserving the structure of giant (extracellular) organelle vesicles released from lysed cells in the hypotonic aqueous medium. Generally, the method of the present invention allows to lower the stretching membrane tension to values less than about ˜2 mN/m to extract and recover GEOVs into the medium. While when the usual stretching membrane tensions known from the state of the art for lysing cells are on average higher than 5 mN/m, all the organelles are fragmented into extracellular nano-vesicles which release their content into the medium and are therefore no longer manipulable unlike claimed GEOVs. Step b) can be carried out by using: mechanical force (e.g. suction pressure, stretching, shearing or acoustic wave), chemical agents and/or detergents, electric field, or laser (light) excitation. For example, the mechanical force is applied with a micro-pipette (radius from about 0.5 to 20 μm) with a suction pressure (generally ranging from about 0.1 to 600 mbar and more preferably from about 5 to 200 mbar (where previous bulk techniques use pressure much larger than 1 bar) that generates the expected stretching membrane tension on cells and leads to the lyse of plasma membrane and giant extracellular organelle vesicles recovery (see example section). For example, after cell swelling and giant intracellular organelle vesicles formation according to step a) of the method of the present invention, applying acoustic field (pressure waves) ranging in the ultrasonic range (preferably from about 16 Khz to 1000 Khz, more preferably from about 20 Khz to 400 Khz) during few seconds (e.g. 0.1 to 100 seconds) allows to generate the expected stretching membrane tension on cells and leading to the lyse of plasma membrane and giant extracellular organelle vesicles recovery (see example section). For example, after cell swelling and giant intracellular organelle vesicles formation according to step a) of the method of the present invention, chemicals working as detergents can be added in a control manner to create the expected stretching membrane tension on cells that leads to the lyse of plasma membrane and giant extracellular organelle vesicles recovery, such as carboxylic acid (with concentrations sufficient to generate plasma membrane lysis), Triton X, Tween, SDS, Brij, Octyl Glucoside, octyl thioglucoside, CHAPS, CHAPSO and others. Using detergents modifies the properties of the giant organelles (see example section).

According to the method of the present invention, giant extracellular organelle vesicles can be recovered from step c) without any labeling and sorted out later by using several non-invasive methods including optical, morphological, mechanical approaches. However according to a particular embodiment of the method of the present invention, the cells of step a) can comprise cells transfected with at least one organelle protein marker or receiving molecules reporting for organelles to facilitate organelle identification. For example, the cells are transfected with at least one organelle protein tagged with a fluorescent or chemical marker used before or after swelling. For example, KDEL marks the ER lumen, Tom20 marks mitochondria, LC3B marks autophagosomes, Lamp1 marks lysosomes and Golgi7 marks the Golgi apparatus.

The present invention also relates to giant extracellular organelle vesicles obtained by a method of the present invention, characterized in that said giant extracellular organelle vesicles:
a) have a luminal volume which is bilayer-bounded; have a surface-to-volume at least lower than 3 μm⁻¹, 2 μm⁻¹, 1.5 μm⁻¹, 1.2 μm⁻¹, 1 μm⁻¹, 0.85 μm⁻¹, 0.75 μm⁻¹, 0.66 μm⁻¹, 0.6 μm⁻¹, 0.5 μm⁻¹, 0.42 μm⁻¹, 0.38 μm⁻¹, 0.33 μm⁻¹, 0.28 μm⁻¹, 0.25 μm⁻¹, 0.2 μm⁻¹, 0.15 μm⁻¹.
b) are produced from organelles of the source cell encompassing endoplasmic reticulum, mitochondria, lysosome, Golgi apparatus, vacuole, chloroplast, autophagosome, autolysosome, endosome, peroxisome, multivesicular bodies.
c) are not surrounded by the plasma membrane of the source cell from which they are produced; meaning that GEOVs are in an extracellular medium.
d) have membrane and lumen composed of proteins, lipids and metabolites. This biochemical composition is a fraction of the total composition of the organelle from which the GEOVs are produced. This fraction is, at least, larger than 0.01%, 0.1%, 0.5%, 1%, 5%, 10%, 20%, 30%, 40%, 50%, 60%, 70%, 90%, 100%.
e) have a volume at least larger than 5 μm³, 15 μm³, 30 μm³, 60 μm³, 100 μm³, 180 μm³, 260 μm³, 380 μm³, 520 μm³, 1000 μm³, 1500 μm³, 2000 μm³, 3000 μm³, 4500 μm³.
f) have a size at least larger than 2 μm, 3 μm, 4 μm, 5 μm, 6 μm, 7 μm, 8 μm, 9 μm, 10 μm, 12 μm, 14 μm, 16 μm, 18 μm, 20 μm in all spatial directions.
g) have none, one, or several contacts with other GEOVs.
h) have none, one, or several contacts with cell components such as cytosol biomolecules, microtubules, actin, filaments, cell nucleus, lipid droplets, ribosomes, centrioles, peroxisomes, plasma membrane and other organelles.

The giant extracellular organelle vesicles of the present invention can be recovered with any type of protein of interest, in contact with different cellular components, or other GEOVs (as shown in FIG. 4A-C). Producing and manipulating for the first time these extracellular complex structures allow to study organelles interactions between cellular components in a new way and the role of intracellular proteins and lipids. New applications in drug discovery, target and biomarker identification could be developed with such GEOV complex structures.

The giant extracellular organelles vesicles of the present invention are functional further characterized in that said GEOVs transport neutral species and ions (membrane transport protein activity that is ranging from at least 0.1%, 1%, 10%, 20%, 30%, 40%, 50%, 60%, 70%, 80%, 90%, 100% of that of the membrane transport activity in a reference cell, e.g., the source cell); comprise protein activity that is ranging from at least 0.1%, 1%, 10%, 20%, 30%, 40%, 50%, 60%, 70%, 80%, 90%, 100% of that of the membrane transport activity in a reference cell, e.g., the source cell. The giant extracellular organelle vesicles of the present invention can be stored, and later delivered for research purposes. The giant extracellular vesicles of the present invention are stable at a temperature of less than 4° C., or −20° C., or −80° C. for at least 1, 2, 3, 6, or 12 hours; 1, 2, 3, 4, 5, or 6 days; 1, 2, 3, or 4 weeks; 1, 2, 3, or 6 months; or 1, 2, 3, 4, or 5 years.

The present invention also relates to a method for screening the activity of a molecule of interest, said method comprising:
a) Contacting at least one type of giant extracellular organelle vesicles of the present invention with the molecule of interest;
b) Measuring the modulation of the activity of the molecule of interest through its interaction with the flux of said at least one type of giant extracellular organelle vesicles.
c) Optionally comparing the activity measured in step b) to the initial activity of the molecule of interest before any contact.

The method for screening of the present invention thus enables measuring the capacity of a molecule of interest to interact with at least one protein on a giant extracellular organelle vesicle; for example screening the efficacy, affinity, selectivity, specificity, toxicity, bio-disponibility, pharmacokinetic, permeability of any drug candidates with at least one protein localized on giant extracellular organelle vesicles obtained through the present invention. Moreover, the method of the present invention can also enable, for example, to modulate the communication of contact between giant extracellular organelle vesicles, to identify molecules targeting the giant extracellular organelle vesicles, to test toxicity of molecules blocking the activity of giant extracellular organelle vesicles. This will thus enable precision medicine and early diagnoses realized on giant extracellular organelle vesicles directly extracted from healthy/sick patient.

The present invention also relates to the use of at least one type of giant extracellular organelle vesicle according to the present invention, for screening the activity of molecules of interest. The interaction or the impact of compounds such as drugs, proteins (e.g. involved in a disorder), or ions on said at least one type of giant extracellular organelle vesicle of the present invention and the contact and communication between them can thus be assessed before the generation and recovery of giant extracellular organelle vesicles. The giant extracellular organelle vesicles of the present invention can thus be used for studying biochemical reactions, or for disease's diagnosis for example by defining patterns of diseases from sick patients and comparing them with healthy patients.

The present invention also relates to the use of at least one type of giant extracellular organelle vesicle according to the present invention as a carrier, thus enabling encapsulation or sequestration of a molecule of interest, e.g. an exogeneous molecule or a molecule/protein expressed by the cell prior to the generation of the giant extracellular organelle vesicles, for drug delivery applications.

### BRIEF DESCRIPTION OF THE FIGURES

FIG. 1 represents (A). Top. Confocal microscopy snap of a Cos 7 cell over-expressing a fluorescent plasma membrane protein (GPI-mcherry) where the nucleus was probed with Hoetsch. Bottom. Confocal microscopy snap of a swollen Cos 7 in contact with the hypotonic aqueous medium of step a); The cell has the same transfection as above. (B). Confocal microscopy snap of a Cos 7 cell over-expressing fluorescent both volumic ER protein (RFP-Kdel) and a membranar mitochondria protein (Mfn2-YFP) before step a). (C). Confocal microscopy snap of a swollen Cos 7 cell over-expressing fluorescent both volumic ER protein (RFP-Kdel) and a membranar mitochondria protein (Mfn2-YFP) after step a). These snaps clearly showed the formation of giant (intracellular) organelle vesicles (GIOVs): here Giant Intracellular Organelle Vesicle of endoplasmic reticulum (ER-GIOV) and Giant Intracellular Organelle Vesicle of Mitochondria (Mito-GIOV) are visualized; Some have a surface-to-volume ratios lower than 0.6 μm⁻¹ (larger than 10 μm in size). (D). Rectangular panel to differentiate each type of GIOVs that are generated from following organelles: ER (ER-GIOVs), mitochondria (Mito-GIOVs), Golgi apparatus (Golgi-GIOVs), autophagosome/autolysosome (Auto-GEOVs), lysosome (Lyso-GEOVs), endosome (Endo-GIOVs), multi-vesicular body (MVB-GIOVs). The nucleus is not considered as a GIOV. (E). Plot of the average GIOVs diameter for each type of organelles after step a) without adding chemicals. Related to FIG. 1D. Mean+/−standard deviation; n report the number of organelles analyzed for each condition. All GIOVs analyzed were spherical. This shows the formation of GIOVs having spherical size larger than 3 μm (e,g, having a surface-to-volume ratio lower than 2 μm⁻¹). The nucleus is not considered as a GIOV. (F). Plot of the relative frequency distribution of GIOV diameter depending on organelles type. Related to FIG. 1D-E. The nucleus is not considered as a GIOV.

FIG. 2 represents (A). Confocal microscopy snap of a swollen Cos 7 cell over-expressing following fluorescent proteins: RFP-Kdel (volumic marker of ER-GIOVs), mcherry-TOMM20-N (membranar marker of Mito-GIOVs) and P58-GFP reporting for ER-GIOVs (P58 signal with RFP-Kdel) and Golgi-GIOVs (Giant Intracellular Organelle Vesicles of Golgi) (P58 signal without RFP-Kdel) after step a). (B). Confocal microscopy snap of a swollen Cos 7 cell over expressing same proteins as in FIG. 2A, incubated with a chemical (nocodazole) 1 h before and during (combined with the hypotonic medium) step a). Adding nocodazole, before and during step a), allows to disrupt microtubules organization, which leads to the production of larger ER-GIOVs in average. (C). Visualization of Giant Extracellular Organelle Vesicles of ER (ER-GEOVs) produced after step c). Overexpressing mCherry-climp63 protein in cos 7-cells before step a) allows to produce larger ER-GEOV (>20 μm in size i.e., with a surface-to-volume ratio smaller than 0.3 μm⁻¹). (D). Rectangular panel showing the different marker used to differentiate three different conditions tested in step a), leading to different size distributions of ER-GIOVs after step c). (E). Plot of the average diameter of ER-GIOVs produced after step c), for each condition of panel 2D. Mean+/−standard deviation; n report the number of vesicles analyzed for each condition. (F). Plot of the relative frequency distribution of ER-GIOVs diameter for each condition of panel 2D. (G). Rectangular panel defining two different types of Mito-GIOVs produced after step c), i.e., recovered from cell over-expressing mcherry-TOMM20-N or Mfn2-YFP. (H). Plot of the average Mito-GIOVs diameter for both conditions of FIG. 2G. Mean+/−standard deviation; n report the number of vesicles analyzed for each condition. Over-expressing Mfn2-YFP (a mitochondria protein implicated in mitochondria contact sites and fusion) allows to produce more ER-GIOVs larger than 5 μm (i.e. surface-to-volume ratio smaller than 1.2 μm⁻¹). (I). Plot of the relative frequency distribution of Mito-GIOV diameter (related to FIG. 2G-H).

FIG. 3 represents (A). Confocal microscopy of GEOVs deriving from different organelles (Fluorescence reporter: ER-GEOV: RFP-Kdel; Mito-GEOV: mcherry-TOMM20-N; Endo-GEOV: pmCherry-2X-FYVE; Golgi-GEOV: P58-GFP; Lyso-GEOV: pMRXIP Lamp1-Venus; Auto-GEOV: LC3-EGFP) produced after step c). After these steps, we also obtain swollen nucleus (Hoestch 33342) and plasma membrane vesicles (GPI_2xmCherry) that are shown. This panel shows GEOVs of each type larger than 5 μm in size (i.e. a surface to volume). (B). Mathematical formula derived from Laplace law to compute the membrane surface tension of a cell or GEOV, that can be applied, for example, with micropipettes to trigger membrane lysis. AP corresponds to the suction pressure imposed into the micropipette, Rₚ corresponds to the radius of the suctioned membrane portion of a GEOVs (in this case, this corresponds to the micropipette radius), R_{Vesicle} corresponds to the radius of the GEOV. (C). Plot of the membrane tension applied to trigger GEOV and plasma membrane vesicle lysis. These measurements have been performed on GEOV and plasma membrane vesicles produced after step c).

FIG. 4 represents (A). Confocal microscopy snaps of GEOVs produced after step c) from Cos 7-cells over-expressing different type of proteins of interest: Top. ER-GEOV covered by YFP-seipin and sec61β-mCherry. Middle. ER-GEOV covered by dGAT1-EGFP and sec61β-mCherry. Bottom. Mito-GEOV covered by Mfn2-YFP and mcherry-TOMM20-N and recovered in contact with ER-GEOVs. (B). Confocal microscopy snaps where GEOVs are produced in contact with other GEOVs or cellular component: Top. An ER-GEOV (sec61β-mcherry) produced and isolated in contact with a lipid droplet (tagged with Bodipy-FL) is shown. Middle. An ER-GEOV (RFP-Kdel) produced and isolated in contact with a Mito-GEOV (mcherry-TOMM20-N) is shown. Bottom. An ER-GEOV (RFP-Kdel) produced and isolated in contact with a peroxisome. (C). Confocal microscopy snaps of ER-GEOVs (looking like semi-spherical vesicles) produced and isolated in contact with a nucleus (Hoestch 33342) recovered after step c), revealing large contacting area between these organelles. These ER-GEOVs are a particular case because there are not spherical but they still are bilayer-bonded and extracellular with a surface-to-volume ratio smaller than 2 μm⁻¹ in contact with the nucleus.

FIG. 5 represents (A). Snaps of an ER-GEOV (sec61β-mcherry) produced after step c), and then fed with OleoylCoA (complemented with NBD-OleylCoA) and DiAcylGlycerol (DAG) at 37° C. As the ER-GEOV has dGATs enzymes on its membrane (Related to FIG. 4A, middle), triglycerides synthesis is visualized on the ER-GEOV membrane among, time thanks to the incorporation of OleylCoA-NBD signal in the ER-GEOV. The following of this signal allows to quantify ER-GEOV functionality through ER protein activity. (B). Plot of the fluorescence signal of OleoylCoA-NBD in the ER-GEOV membrane during the feeding experiment (Related to FIG. 5A).

FIG. 6 represents (A). Confocal microscopy snap of swollen Cos 7 cells after step a). Cells are spheric, entire and their plasma membrane is intact. A lot of GIOV trapped inside the swollen cells can be visualized. (B). Confocal microscopy snap of Cos 7 cells after step a), and the addition of detergent (Oleic acid) combine with fluorescent detergents as a step b). The addition such of detergents after a) allow the production of GEOVs in the bulk thanks to the breakage of plasma membrane and the release of GEOVs. GEOV's membrane also incorporate the detergents, which can modify their compositions, properties, biophysical parameters of membranes and both folding and activity of proteins. (C). Confocal microscopy snap of Cos 7 cells after step a) step b) which has been performed by submitting plasma membrane to an acoustic field. Precisely, the cells were subjected 5 seconds to ultrasonication (acoustic wave of ˜40 Khz). This acoustic field allows the release of some ER-GEOVs (reported with RFP-Kdel) in the bulk. Some cells are still entire, meaning that precise control of ultrasonication parameter is crucial to recover GEOVs without broking them.

FIG. 7 represents confocal microscopy snap of a swollen cos 7 cell after step a). The step a) has been performed by changing the osmolarity of the hypotonic aqueous buffer sequentially from 150 mOsm, to 75 mOsm, to 30 mOsm, to 20 mOsm in the end. Cells were incubated 5 minutes with each medium and imaged in contact the last one. Before step a), cells were transfected to visualize ER-GIOV (RFP-Kdel and kde-EGFP) and Mito-GIOV (mcherry-TOMM20-N). Cell and internal organelle vesicles do not swell efficiently: Some ER-GIOV are not spherical and some are very small (Mito-GIOV are not larger than 3 μm). These step a) of sequential swelling do not lead to large GIOV and spheric cell formation (that are easier to lyse). This shows that modulating the step a) is really important for the production of GEOVs.

FIG. 8 represents A) Plot of the plasma membrane lysis tension of entire swollen cells (containing GIOVs) subjected to step a) versus cells subjected to steps a)+a′). The tension in step b) has been applied during less than 59 seconds. Mean+/−standard deviation are shown. B) Relative frequency distribution of plasma membrane lysis tension event for entire swollen cells (containing GIOVs) subjected to only step a). The tension in step b) has been applied during less than 59 s. This plot shows that cells have a large bimodal distribution. Some cells have a lysis tension under 2 mN/m and other have lysis tension larger than 5 mN/m (in the range of the prior art). These last are difficult to lyse with gentle conditions without breaking GEOVs (because of their lysis tension value as shown in FIG. 3C). C) Relative frequency distribution of plasma membrane lysis tension event for entire swollen cells subjected to both steps a) and a′) (with the same step a)). The tension in step b) has been applied during less than 59 s. The large majority of swollen cells have very low lysis tensions under 2 mN/m (More than 50% of the cells have lysis tension under 0.75 mN/m with these conditions). Which is lower than the lysis tension value of almost all GEOVs (FIG. 3C). Step a′) allows to facilitate the lysis of the cell and to recover larger and more GEOVs after step c).

FIG. 9 represents A) Confocal microscopy snaps of ER-GEOVs produced after step a) (Incubation=13 min), a′), b) and c). These ER-GEOVs exhibit sizes larger than 10 μm (i.e. surface-to-volume ratio smaller than 0.6 μm⁻¹), and are extracted from only one cell. Modulating step a) with a brutal osmotic shock, adding step a′), and, reducing the applied tension of step b), finally allows to produce all these larger ER-GEOVs from one single cell.

### EXAMPLES

### Example 1: Method for Generating Giant Organelle Vesicles and Collecting the Same: Using Mechanical Force (Suction Pressure) after Swelling

### Cell Culture

Cos 7 mammalian cells were maintained in Dulbecco's Modified Eagle Medium (DMEM) supplemented with 10% heat inactivated FBS and 1% penicillin-streptomycin. Before the GEOVs production protocol, cells were cultivated 48 h in DMEM media at 37° C. with 5% CO₂. Moreover, cells were transfected 24 h with the indicated plasmids to probe organelle before step a), GIOV after step a) and GEOVs after step c) of the method of the present invention. Cells were cultured in adhering dishes, pre-treated or not with adhesion agents.

### Cell Transfection Reporting for Giant Extracellular Organelle Vesicle

Cells were transfected with different plasmids fused with fluorescent protein constructs (e.g., eGFP or mCherry) 24 h before GEOV production. These plasmids serve to express proteins reporting for different GEOVs. Kdel and Sec61β plasmids were used to identify the endoplasmic reticulum, Tom20 and/or MitofusinII plasmids to identify mitochondria, GP58 or Kde for the Golgi, FYVE for endosomes, LC3 for autophagosome and Lamp1 for the lysosomes.

### Giant Intracellular Organelle Vesicle Formation and Size Distribution Measurements

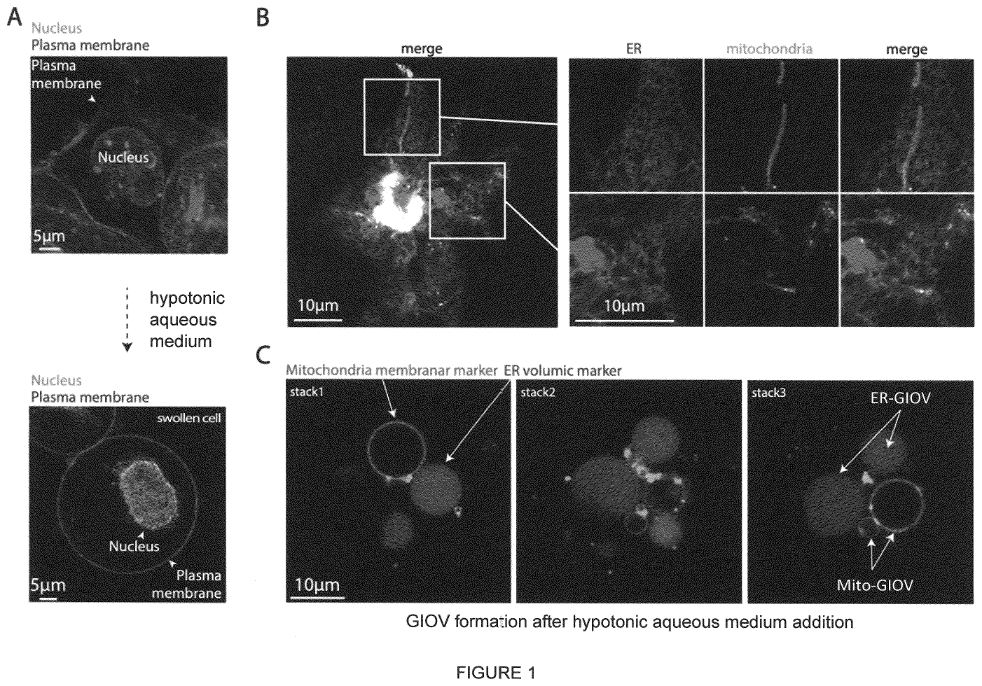

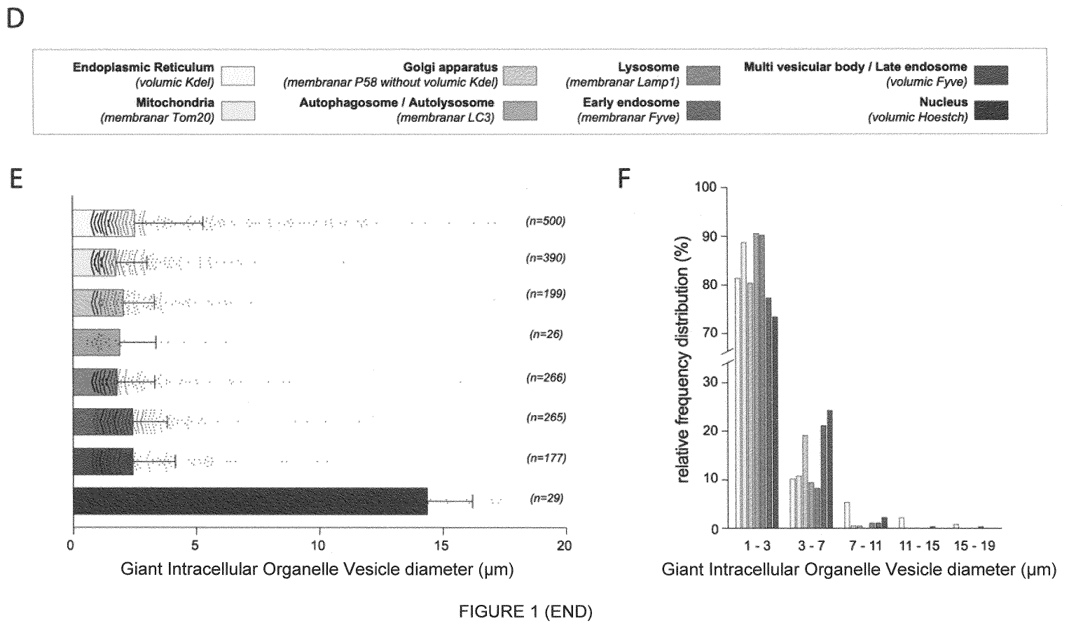

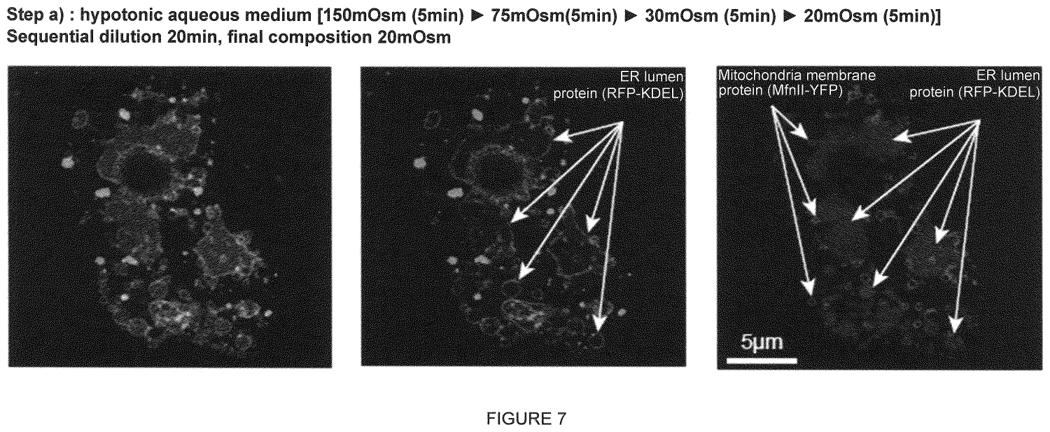

For giant intracellular organelle vesicle formation, before cells were confluent, the cell culture media was diluted with H₂O, pH 7.4, at 37° C. and 5% CO₂. See the swollen cells after step a), i.e., after swelling and before GEOVs production (FIG. 1C). The osmolarity gradient of the swelling solution was controlled and was adjusted before and/or during step a) depending on the cell type (from 100 mOsm/L to 0.1 mOsm/L). During step a), osmotic gradient can also be changed during an amount of time, and, modulated (FIG. 7A), to control size distribution of GIOVs and the cinetic of cell swelling. After cell swelling (step a)), the cell solution was injected in clear hypotonic media on a glass plate, pre-treated with Bovine Serum Albumine (BSA) 10% v/v. Confocal microscopy snaps were then taken on entire swollen cells with different protein markers to identify each type of GIOVs. The diameter of each GIOVs was measured on each stack (1 μm per slice) of these swollen cells to report for size distribution of GIOVs. (FIG. 1E-F). As we are interested on over-micrometric vesicles, only those having a size over 0.75 μm were considered for size distribution measurements.

### Size Control of Giant Extracellular Vesicles

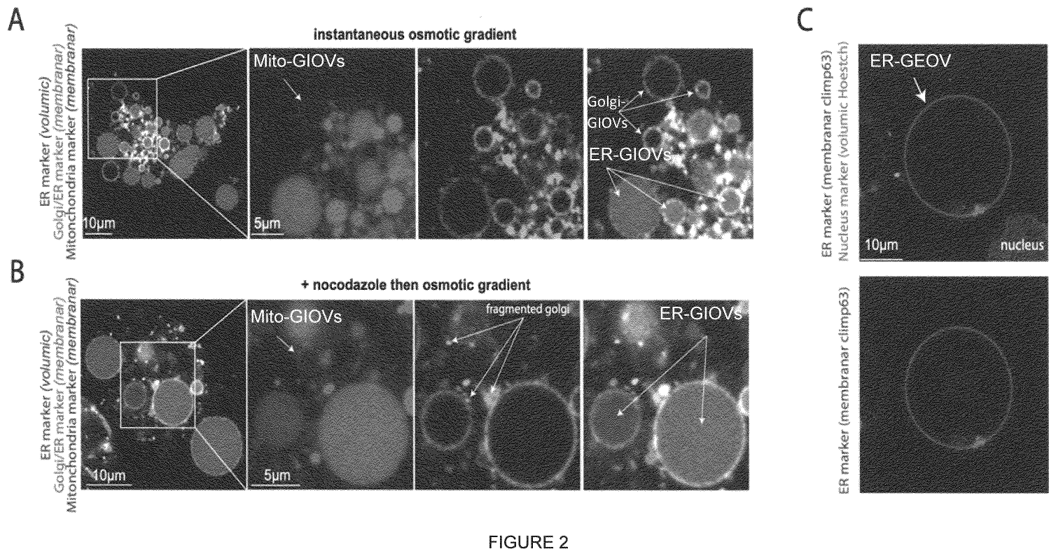

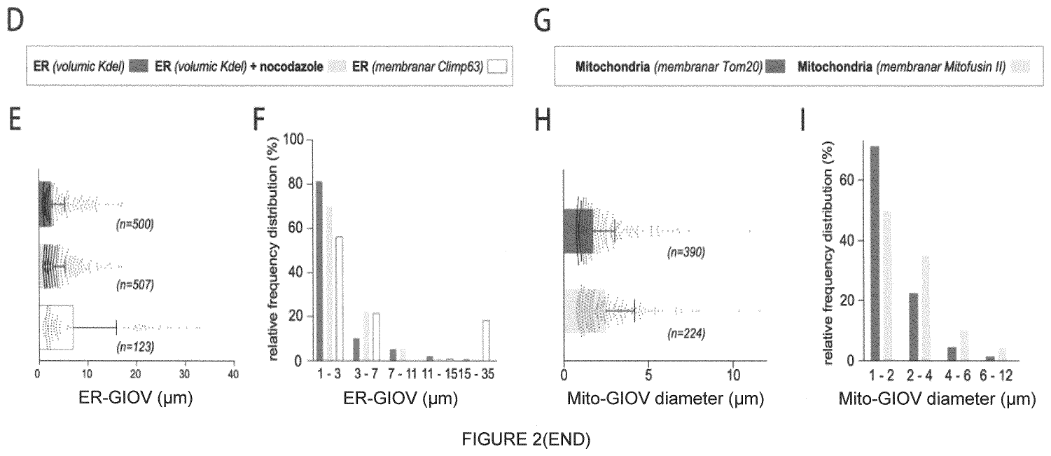

To control the production of GEOVs, the swelling of organelles was carried out with a hypotonic aqueous medium (FIG. 2A). Adding chemical agents to this media allows to control even more the distribution size of GEOVs produced after step c) (FIG. 2).

To increase the size of Giant Endoplasmic Reticulum (ER-GEOV), we added nocodazole at 2.5 μM in the culture media one hour before step a) (FIG. 2B) and during step a). Other conditions are shown where cells were transfected with a ER membrane shaping protein: Climp63 before step a) (FIG. 2C). The protocol with nocodazole allowed to form a higher proportion of Giant ER than without (FIG. 2D-F). Moreover, over-expressing the protein Climp63 in cells allowed the formation of huge ER-GEOV (15 μm-35 μm) (FIG. 2D-F).

Following this idea of over-expressing membrane shaping proteins, the size of Mito-GEOV was also modulated. The mitofusinII protein was overexpressed instead of the TOM20 mitochondria membrane proteins (FIG. 2H vs 2I). Over-expressing the mitofusinII protein allowed to form bigger Mito-GEOV. (FIG. 2G-I).

Step a′) and Viability of Future GEOV Produced.

Under conditions where cells are swollen and subjected to a back-and-forth motion of the hypotonic medium (step a′).

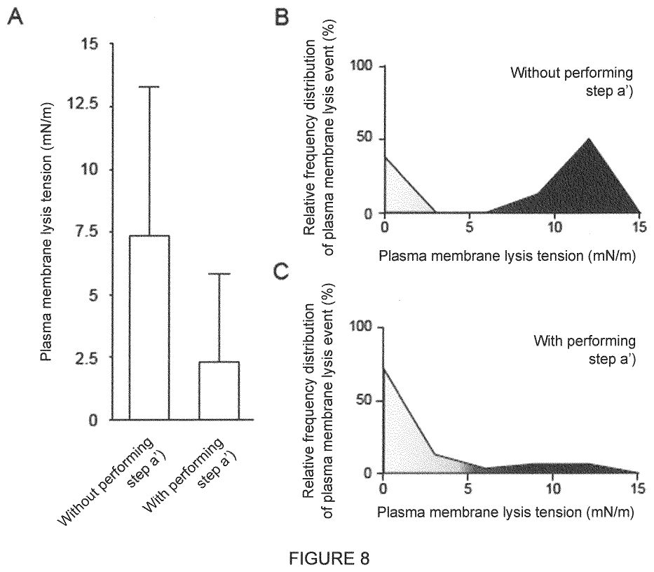

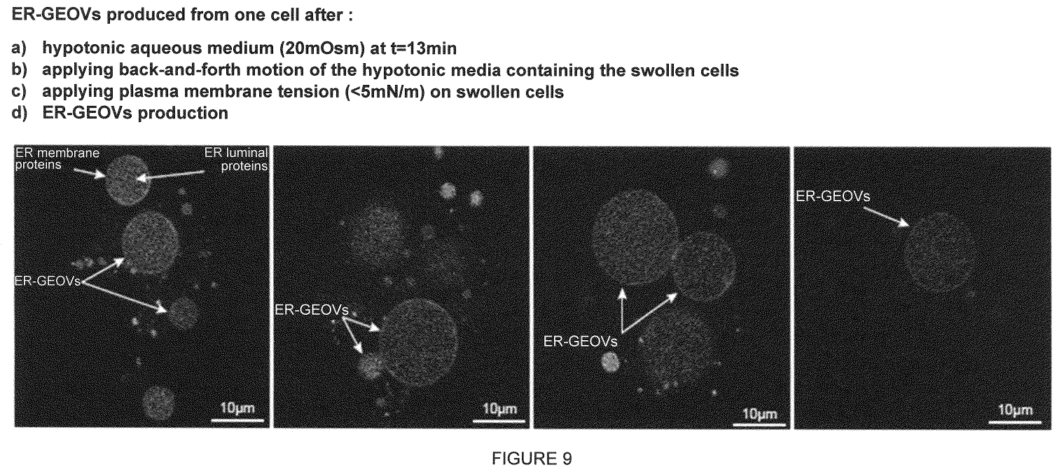

Step a′) is performed during 5 s. The back-and-forth motion is performed on all the volume of the hypotonic aqueous medium 3 times in a row. This step allows to destabilize the cytoskeleton of the cell and to reduce its lysis tension. (FIG. 8) which leads to the production of GEOVs (of better size distribution) without lysing them during plasma membrane lysis and removal (FIG. 9).

### Giant Extracellular Organelle Vesicle and Cell Lysis Tension Measurements:

To determine an estimation of the bilayer lysis tension of giant extracellular organelle vesicles (GEOVs), the micropipette aspiration technique was used. Micropipette radius were around 1 μm. Thanks to a slight aspiration, a bilayer tongue of the GEOV (or the plasma membrane) was sucked into the micropipette. The aspiration was then increased at an approximate rate of 10 mbar/min, causing a proportional increase in the bilayer surface tension. At a certain applied tension, the GEOVs (or plasma membrane) ruptured because of a pore opening. Therefore, the measured lysis tension was taken as the higher tension reached just before bilayer rupture. Using Laplace's law, and the measurement of the pipette inner radius (Rₚ), Vesicle radius (R_{Vesicle}), and suction pressure ΔP, the applied surface tension γ of the interface was calculated:

$$γ = \frac{Δ P \cdot {R}_{p}}{2 \cdot ( 1 - {R}_{p} / {R}_{Vesicle} )}$$

The applied suction pressure was carried out using a syringe. The resulting pressure was measured with a pressure transducer (DP103; Validyne Engineering, North-ridge, CA), the output voltage of which was monitored with a digital volt-meter. The pressure transducer (range 55 kPa) was calibrated before the experiments.

### Controlling the Release of Giant Extracellular Organelle Vesicles Thanks to Cell Lysis and Opening of Plasma Membrane.

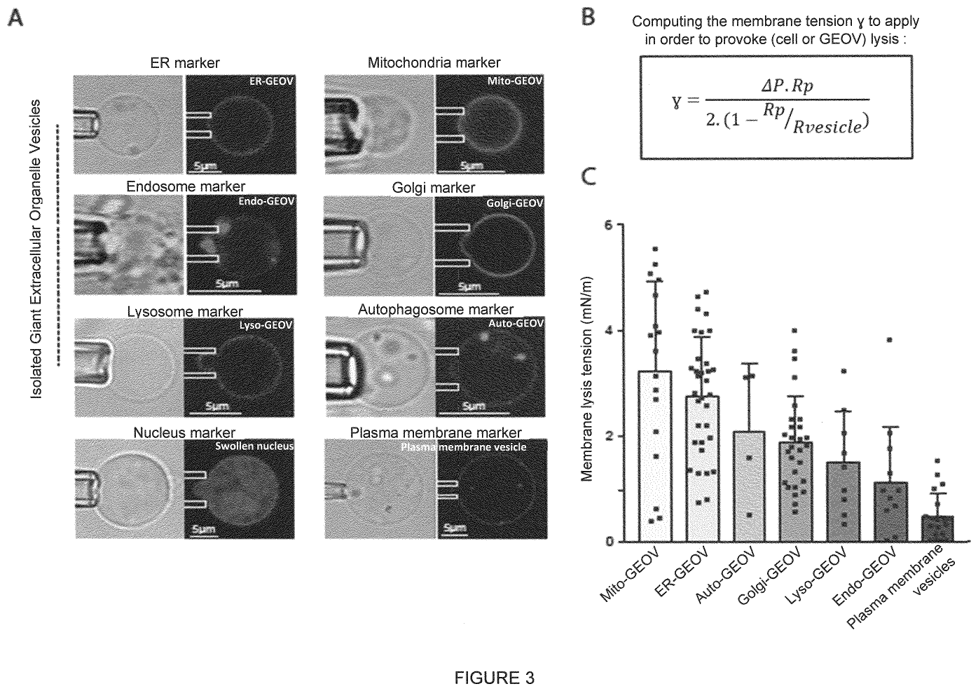

As an example for step b), cells were micro-manipulated under confocal microscope. (data not shown). Giant extracellular organelle vesicles (GEOVs) were observed in the culture media. The giant extracellular organelle vesicles were released thanks to the lysis and the removal of the plasma membrane (data not shown). To quantify the tension to apply on plasma membrane to produce GEOVs release without breaking them, the lysis surface tension of GEOVs, plasma membrane vesicles and entire swollen cells (subjected to a) or a)+a′)) were measured and averaged (FIG. 3C and FIG. 8A-C). In average, entire swollen cells of step a′) have a lysis surface tension significantly lower than entire swollen cells not subjected to step a′) (FIG. 8A-C). After step c), the plasma membrane vesicle lysis tension was found to be much lower than all other GEOV (FIG. 3C). In average, GEOVs have a membrane lysis tension (FIG. 3C and FIG. 8A-C) higher than different type of plasma membrane (Entire swollen cells of a)+a′), some entire swollen cells of a), plasma membrane vesicles of c)). which was subjected to step a′). Finally, combining a) with a′) allows to trigger cell lysis at very low ranges of surface tensions (<0.75 mN/m), for a large lajority of cells. This avoids to trigger GEOV lysis during extraction. Such method allows to recover giant extracellular organelle vesicles, that are functional and stable, in contact or not with other GEOVs or cellular components.

### Giant Extracellular Organelle Vesicles Expressing a Protein of Interest

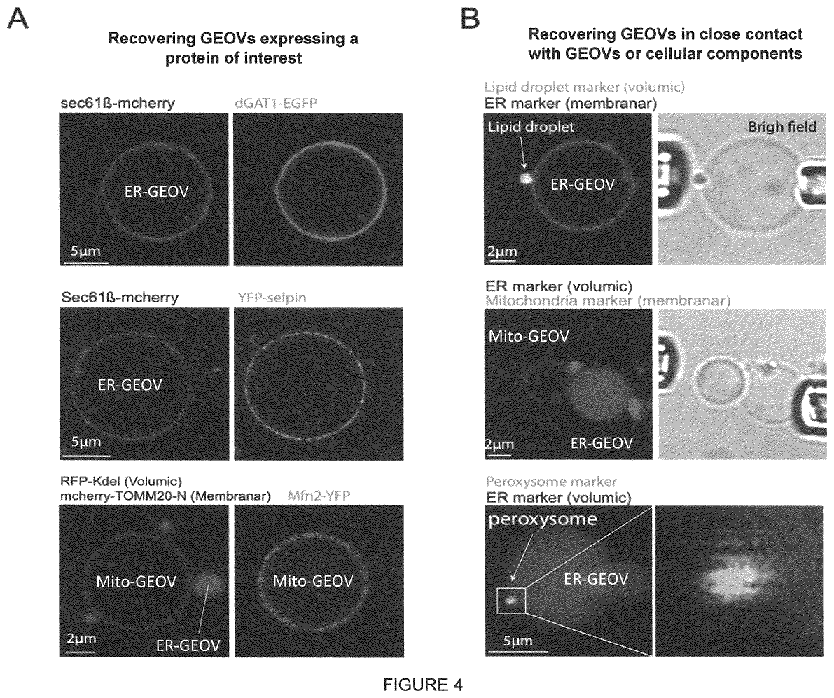

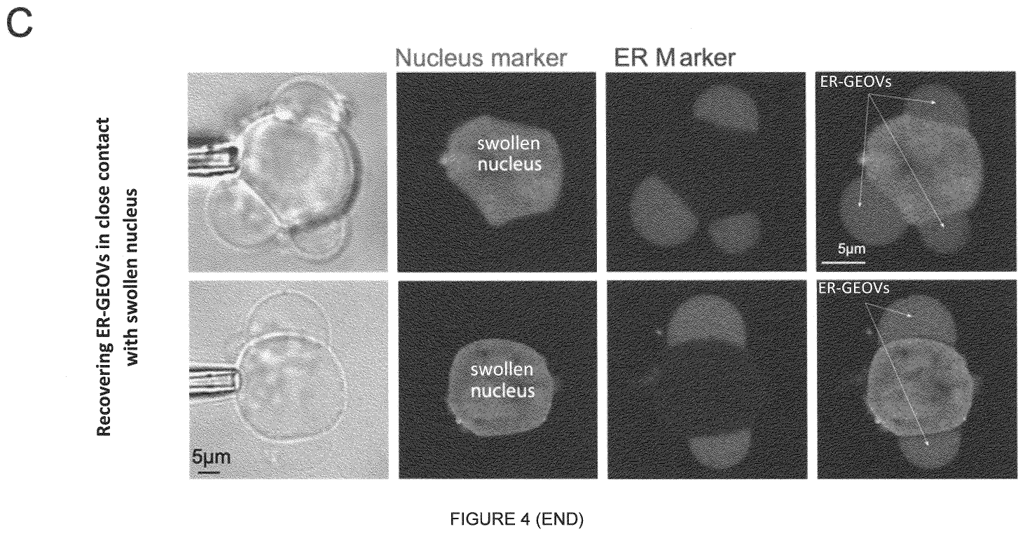

One of the great potentials of the present invention is the production of a GEOV over-expressing a chosen protein, or few chosen proteins (FIG. 4A)

### Giant Extracellular Organelle Vesicles in Contact are Isolated

GEOVs in contact with other GEOVs are also produced after step c), and isolated (such as ER-GEOV/Mito-GEOV contacts and ER-GEOV/lipid droplets) (FIG. 4B). Particular systems were also isolated like the ER-GEOV/nucleus contacts (FIG. 4C).

### Example 2: Method for Generating Giant Extracellular Organelle Vesicles: Using Detergents in Step B) after Swelling

### Giant Intracellular Organelle Vesicles Formation: Same Protocol as in Example 1

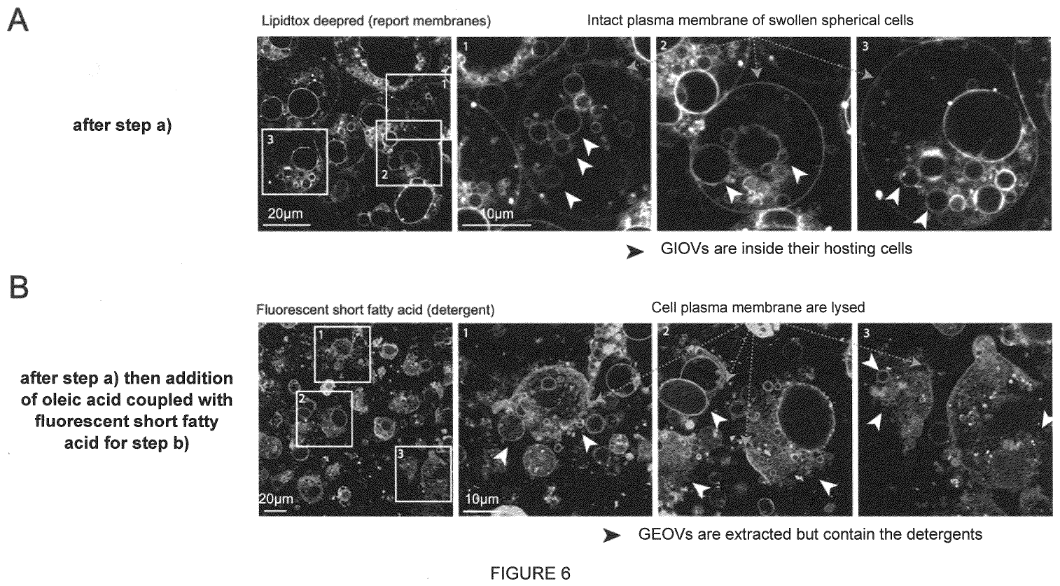

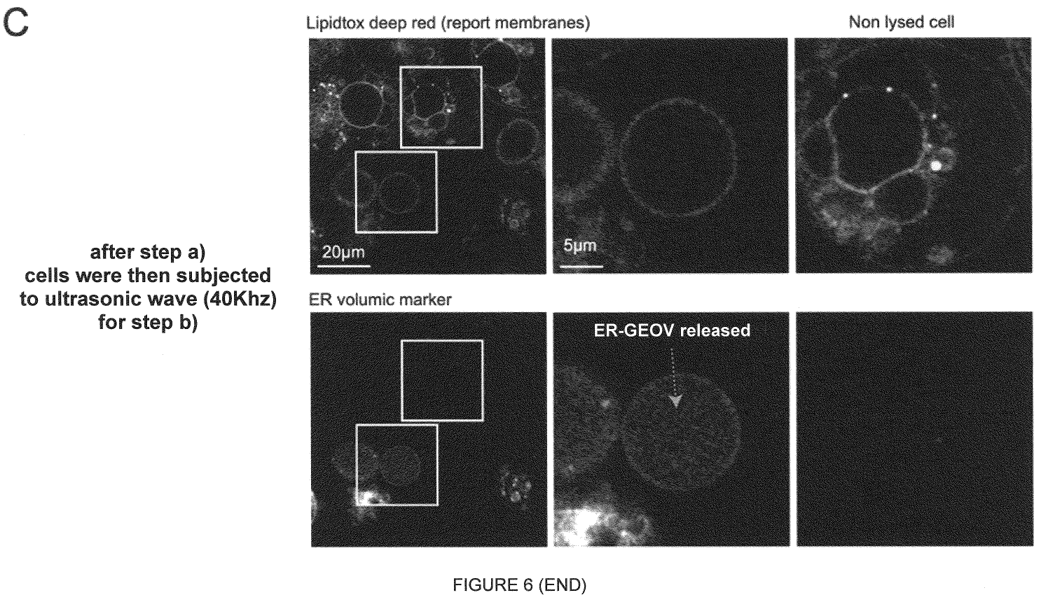

### Controlling the Release of Giant Extracellular Organelle Vesicles Thanks to Cell Lysis by Adding Chemicals or Detergents. (FIG. 6B)

After cell culture, transfection and osmosis, i.e. step a), a chemical which act as a detergent was added. An oleic acid (detergent) solution (solubilized in H₂O with 10% BSA) was added to the swollen cell solution at 400 μM (Bodipy-C₁₂ fluorescent probe was added to visualize the incorporation of oleic acid in membranes). After few minutes, cells were imaged. Giant extracellular organelle vesicles (GEOVs) were produced and visualized outside the cells which exhibit the plasma membrane lysis and removal.

Adding these chemicals on swollen cells with giant (intracellular) organelle vesicles that are trapped inside, allows the extraction and recovery of giant extracellular organelle vesicles (GEOVs). As expected, the membranes of GEOVs are modified due to accumulation of detergents inside their membranes (FIG. 6B).

### Example 3: Method for Generating Giant Extracellular Organelle Vesicles and Collecting the Same: Using Ultrasonic Wave after Swelling (FIG. 6c)

### Giant Intracellular Organelle Vesicles Formation: Same Protocol as in Example 1

### Controlling the Release of Giant Extracellular Organelle Vesicles Subjecting Cells to an Acoustic Field

After cell culture, transfection and osmosis, i.e. step a), the swollen cell solution was subjected to an acoustic field in a bath sonicator during few seconds, to apply plasma membrane surface tensions perturbations and trigger cell lysis. A 40 kHz ultrasonic frequency was used to lyse the cells and release giant extracellular organelle vesicles (GEOVs) in the bulk.

### Example 4: Giant Extracellular Organelle Vesicles Functionality

At this step the functionality of recovered giant extracellular organelle vesicles (GEOVs) was studied regarding both assimilation and transformation of oleyl-CoA by proteins of GEOVs.

Triglyceride Synthesis. Oil Synthesis in ER Giant Extracellular Organelle Vesicle (ER-GEOV)

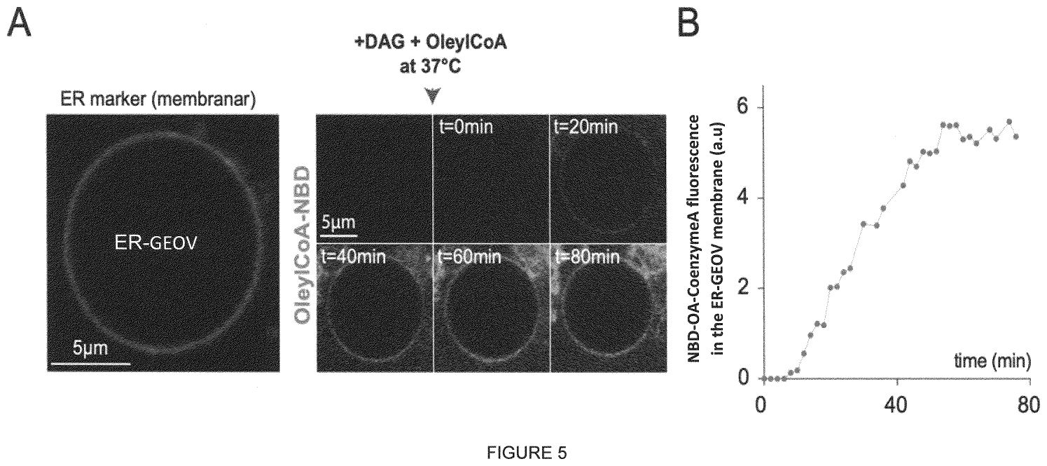

Triglyceride synthesis: A mixture of diolein at 8 mM, Oleyl-CoA at 2 mM (+NBD-Oleyl-CoA at 20 μM) complexed with BSA (0.5 wt %) in hypotonic culture media (DMEM: H₂O 5:95) was prepared before the experiment. After their production (i.e. after step c), ER-GEOV were incubated during 30 minutes at 37° C. and 5% CO₂ with the addition of 50 μL of the feeding mixture. The final concentrations were 200 μM, 50 μM and 0.5 μM respectively for the diolein, Oleyl-CoA and NBD-Oleyl-CoA in 2 mL of hypotonic culture media. For FIG. 5A, the incorporation of oleic acid in ER-GEOV was monitored thanks to the fluorescence signal of NBD-Oleyl-CoA in the ER-GEOV membrane. Triglycerides, diglycerides and phospholipids synthesis was identified and quantified by Thin Layer Chromatography (TLC) and Mass Spectrometry (MS) analysis (Data not shown).

### Equipment

All micrographs were made on a Carl ZEISS LSM 800. Glass coverslips were from Menzel Glaser (24-36 mm #0). Micropipettes were made from capillaries (1.0 OD 0.58 ID 150 Lmm 30-0017 GC100-15b; Harvard Apparatus) with a micropipette puller (model P-2000; Sutter Instruments). Micromanipulation was performed with TransferMan 4r (Eppendorf). Pressure measurement unit (DP103) was provided by Validyne Engineering.

1. A method for production of giant extracellular organelle vesicles from cells, said method comprising:
a) Contacting the cells during 0.5 to 30 minutes with an hypotonic aqueous medium with an osmolarity ranging from 0.1 to 100 mOsm/L;
b) Applying a membrane tension on cells ranging from 10⁻³ to 5 mN/m during 10⁻⁴ to 100 seconds; and
c) Collecting the giant extracellular organelle vesicles into the hypotonic aqueous medium.

2. The method according to claim 1, further comprising, after step a) and before step b), a step a′) of generating a back-and-forth motion of the hypotonic aqueous medium to displace cells at a speed ranging from 0.01 m/s to 10 m/s during 0.01 seconds to 10 minutes.

3. The method according to claim 1, wherein the hypotonic aqueous medium comprises one or more molecules chosen from the group consisting of: nocodazole, trypsin, latrunculins, misakinolides, mycalolides, aplyronides, vinblastine, rotenone, swinholides, jasplakinolides, vincristine, demecolcine, cytochalasins, colchicine, vinca-alcaloids, dihydropyridine, phenylalkylamine, benzothiazepine, gabapentinoids, blebistatin, benzytoluen sulphonamide, butanediome monoxime, thaspsigargin, xelospongin, Triton X, Tween, SDS, Brij, Octyl Glucoside, octyl thioglucoside, CHAPS, CHAPSO, magnesium.

4. The method according to claim 1, wherein the cells from which the giant extracellular organelle vesicles are produced, are cultured on a support or in bulk, or are recovered from tissues, organs, organoids or organisms.

5. The method according to claim 1, wherein step c) generates giant extracellular organelles vesicles having a surface-to-volume ratio from 3 μm⁻¹ to 0.15 μm⁻¹.

6. The method according to claim 1, wherein step b) is carried out using mechanical force, chemical agents, detergents, electric or acoustic field, and laser excitation.

7. The method according to claim 1, wherein the cells of step a) and b) are previously transfected with at least one organelle protein marker or receiving molecules reporting for organelles.

8. Giant extracellular organelle vesicles obtained by a method according to claim 1, characterized in that said giant extracellular organelle vesicles have a surface to-volume ratio from 3 μm⁻¹ to 0.15 μm⁻¹.

9. The giant extracellular organelle vesicles according to claim 8 characterized in that said giant extracellular organelle vesicles have a lumen, are bilayer-bounded and are free from the plasma membrane of the hosting cell.

10. The giant extracellular organelle vesicles according to claim 8, chosen from the group consisting of endoplasmic reticulum, mitochondria, lysosome, Golgi apparatus, vacuole, chloroplast, autophagosome, autolysosomes, endosomes, peroxisome, multivesicular bodies, plastids.

11. A method for screening the activity of a molecule of interest, said method comprising:
a) Contacting a flux of at least one type of giant extracellular organelle vesicle according to claim 8 with the molecule of interest;
b) Measuring the activity of the molecule of interest through its interaction with the flux of said at least one type of giant extracellular organelle vesicles; and
c) Optionally comparing the activity measured in step b) to the initial activity of the molecule of interest before any contact.

12. A use of at least one type of giant extracellular organelle vesicle according to claim 8, for screening the activity of a molecule of interest.
